(19) United States
(12) Patent Application Publication
(10) Pub. No.: US 2024/0285408 A1
(43) Pub. Date: Aug. 29, 2024

(54) SURGICAL TETHER PROTECTION DEVICE

(71) Applicant: EDWARDS LIFESCIENCES CORPORATION, Irvine, CA (US)

(72) Inventors: Jyoti B. Rao, Brea, CA (US); Devin D. Nguyen, Garden Grove, CA (US)

(21) Appl. No.: 18/657,316

(22) Filed: May 7, 2024

Related U.S. Application Data
(63) Continuation of application No. PCT/US2022/050329, filed on Nov. 17, 2022.
(60) Provisional application No. 63/264,639, filed on Nov. 29, 2021.

(51) Int. Cl. A61F 2/24; A61F 2/00

(52) U.S. Cl. CPC A61F 2/2487; A61F 2002/0081; A61F 2220/0033; A61F 2220/0075; A61F 2230/0006; A61F 2230/0065; A61F 2230/0069; A61F 2250/0003

(57) ABSTRACT

A tether protection device can comprise an inflatable member configured to be positioned over an opening in a target tissue. The inflatable member can comprise a central portion defining an opening extending through the inflatable member, the opening of the inflatable member being configured to allow for a plurality of tethers extending from the opening in the target tissue to be disposed at least partially therein. A fluid port can be associated with the inflatable member, the inflatable member being configured to be inflated via the fluid port to an initial inflation state in which the plurality of tethers is allowed to move freely within the opening of the inflatable member, and a first inflation state in which at a portion or the central portion is collapsed around and in contact with the plurality of tethers.

### CROSS REFERENCE TO RELATED APPLICATIONS

This application is a continuation of International Patent Application No. PCT/US2022/050329, filed Nov. 17, 2022, which claims the benefit of U.S. Patent Application No. 63/264,639, filed Nov. 29, 2021, the entire disclosures all of which are incorporated by reference for all purposes.

### BACKGROUND

The present disclosure generally relates to the field of tether protection devices used in proximity to tissue openings. Surgical tethers can be anchored to a target tissue as a part of medical procedures for treating various health conditions. Anchoring surgical tethers to the heart wall can be a part of heart valve repair procedures to address a number of heart valve abnormalities. The surgical tethers can be anchored to the heart wall so as to maintain a desired tension in the surgical tethers.

### SUMMARY

Described herein are systems, devices and methods relating to one or more tether protection devices comprising an inflatable member. The inflatable member can have a central portion defining an opening extending through the inflatable member. The opening in the inflatable member can be configured to receive respective portions of one or more tethers. The inflatable member can be configured to be maintained in different pressurized states. For example, the inflatable member can be configured to be pressurized to a first inflation state such that at least a portion of the central portion collapses around portions of the one or more tethers disposed within the opening of the inflatable member, holding the one or more tethers in place. In some instances, the tether protection devices can be used in heart valve repair procedures that use one or more tethers to anchor a heart valve leaflet to a portion of the heart wall. For example, the tether protection device can be positioned at least partially over an opening in a target tissue, such as a portion of an externally oriented surface of the heart wall, such that the opening of the inflatable member has the same or similar orientation as the opening in the target tissue. One or more tethers extending from the opening in the target tissue can be configured to be disposed through the opening of the inflatable member. The tensions of the one or more tethers can be maintained while at least a portion of the central portion of the inflatable member is collapsed around respective portions of the tethers, without damaging and/or breaking the tethers.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the disclosure. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 is a cross-sectional view of a human heart and a tether deployed to couple a mitral valve leaflet to a left ventricular wall.

FIG. 2 is a cross-sectional view of a human heart and a tether deployed to couple a mitral valve leaflet to a left ventricular wall, where a tether protection device is used to facilitate coupling of the mitral valve leaflet to the left ventricular wall.

FIG. 3A is a top-down view of an example of a tether protection device comprising an inflatable member in an inflated state. FIG. 3B is a top-down view, and FIG. 3C is a perspective view, showing the inflatable member of FIG. 3A in another inflated state.

FIGS. 4A and 4B are top-down views showing the inflatable member of FIG. 3A in different inflated states.

FIG. 5A is a top-down view, and FIG. 5B is a perspective view, of an example of a tether protection device comprising an inflatable member that has a central portion comprising a plurality of bumps thereon.

FIG. 6A is a top-down view, and FIG. 6B is a perspective view, of an example of a tether protection device comprising an inflatable member that has a central portion comprising a plurality of ridges thereon.

FIG. 7A is a top-down view, and FIG. 7B is a cross-sectional view, of an example of a tether protection device comprising an inflatable member that has a central portion comprising a wall thickness that is less than that of a circumferential portion of the inflatable member.

FIG. 8A is a top-down view, and FIG. 8B is a cross-sectional view, of an example of a tether protection device comprising an inflatable member that has a central portion defining an opening having an elongate shape.

FIG. 9A is a top-down view, and FIG. 9B is a cross-sectional view, of an example of a tether protection device comprising an inflatable member that has a central portion which defines an opening having a tubular lumen configuration.

FIG. 10A is a top-down view, and FIG. 10B is a perspective view, of an example of a tether protection device comprising an inflatable member and a ring member disposed within an opening of the inflatable member. FIG. 10C is a top-down view of the inflatable member where the ring member is removed from the opening of the inflatable member.

FIG. 11 is a process flow diagram of an example of a process to deploy a plurality of tethers that involves using a tether protection device as described herein.

### DETAILED DESCRIPTION

The headings provided herein are for convenience only and do not necessarily affect the scope or meaning of the claims.

In some examples, the present disclosure relates to systems and methods for tether protection devices comprising an inflatable member having an opening extending therethrough. The inflatable member can be configured to be positioned over an opening in a target tissue such that one or more tethers extending from the opening in the target tissue can be advanced through the opening of the inflatable member. A portion of the inflatable member can collapse around portions of the one or more tethers and pinch the portions of the one or more tethers. The inflatable member can hold the one or more tethers in their respective positions, thereby facilitating securing the one or more tethers to the target tissue.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

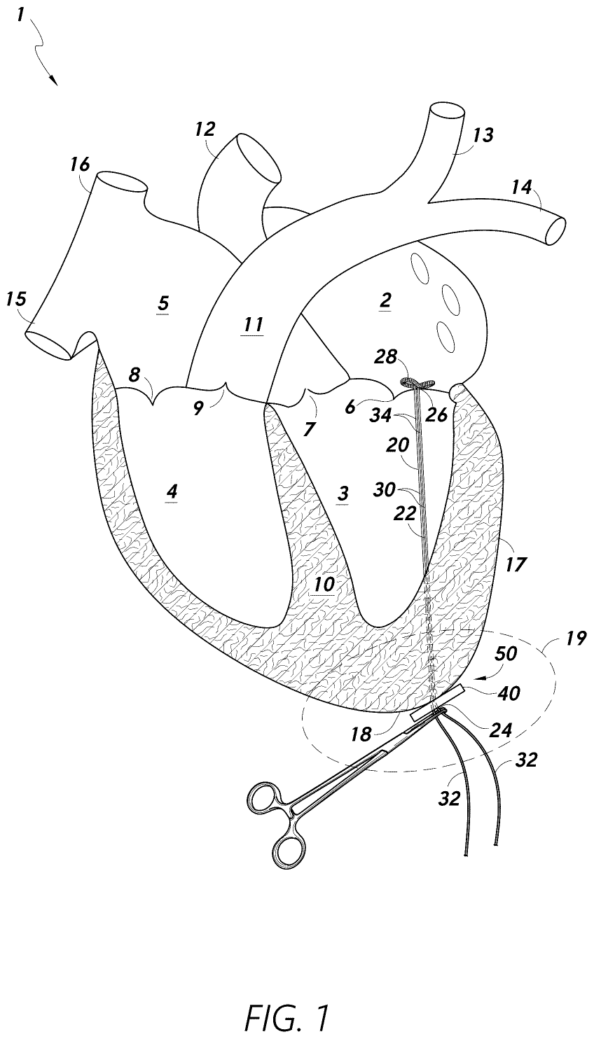

FIG. 1 shows a cross-sectional view of a human heart 1 and a tether deployed to couple a leaflet of a mitral valve 6 to a ventricular wall 17 of a left ventricle 3. The heart 1 includes four chambers, namely the left atrium 2, the left ventricle 3, the right ventricle 4, and the right atrium 5. A wall of muscle, referred to as the septum 10, separates the left atrium 2 and right atrium 5, and the left ventricle 3 and right ventricle 4. Blood flow through the heart 1 is at least partially controlled by four valves, the mitral valve 6, aortic valve 7, tricuspid valve 8, and pulmonary valve 9. The mitral valve 6 separates the left atrium 2 and the left ventricle 3 and controls blood flow therebetween. The aortic valve 7 separates and controls blood flow between the left ventricle 3 and the aorta 12. The tricuspid valve 8 separates the right atrium 5 and the right ventricle 4 and controls blood flow therebetween. The pulmonary valve 9 separates the right ventricle 4 and the pulmonary trunk or artery 11, controlling blood flow therebetween.

In a healthy heart, the heart valves can properly open and close in response to a pressure gradient present during various stages of the cardiac cycle (e.g., relaxation and contraction) to at least partially control the flow of blood to a respective region of the heart and/or to blood vessels. Deoxygenated blood arriving from the rest of the body generally flows into the right side of the heart 1 for transport to the lungs, and oxygenated blood from the lungs generally flows into the left side of the heart 1 for transport to the rest of the body. During ventricular diastole, deoxygenated blood arrive in the right atrium 5 from the inferior vena cava 15 and superior vena cava 16 to flow into the right ventricle 4, and oxygenated blood arrive in the left atrium 2 from the pulmonary veins to flow into the left ventricle 3. During ventricular systole, deoxygenated blood from the right ventricle 4 can flow into the pulmonary trunk 11 for transport to the lungs (e.g., via the left 14 and right 13 pulmonary arteries), and oxygenated blood can flow from the left ventricle 3 to the aorta 12 for transport to the rest of the body.

A number of conditions can contribute to a higher than normal pressure in the left atrium 2. Dysfunction of the mitral valve 6 can contribute to elevated left atrial pressure. Conditions such as mitral valve regurgitation and/or stenosis may result in difficulty in pumping blood from the left atrium 2 to the left ventricle 3, contributing to elevated pressure in the left atrium 2. Valve stenosis can cause a valve to become narrowed or obstructed. Mitral valve stenosis can restrict blood flow from the left atrium 2 to the left ventricle 3. Valve regurgitation occurs when a valve does not close properly. For example, regurgitation can occur due to improper coaptation of the valve leaflets, such as due to prolapse of one or more of the valve leaflets. Mitral valve regurgitation can result in blood flow leakage back into the left atrium 2 from the left ventricle 3 when the left ventricle 3 contracts. Restricted flow of blood from the left atrium 2 into the left ventricle 3, and blood flow leakage from the left ventricle 3 back into the left atrium 2 can both contribute to elevated atrial pressure. Dysfunction in the left ventricle 3 can also contribute to elevated left atrial pressure. Elevated left atrial pressure may lead to left atrial enlargement, producing symptoms such as shortness of breath during exertion, fatigue, chest pain, fainting, abnormal heartbeat, and swelling of the legs and feet.

Heart valve repair and/or replacement procedures can be performed to improve or restore valve function. In such procedures, an opening 50 can be formed through the heart wall 17 to access a heart valve. For example, mitral valve repair procedures can be performed to alleviate mitral valve dysfunction, including mitral valve prolapse. In some cases, a transapical approach can be used to gain access into the heart 1. For example, mitral valve repair procedures can include accessing the mitral valve 6 from within the left ventricle 3, where entry into the left ventricle 3 can be achieved through the left ventricular wall in the apical region 19 of the heart 1. The heart wall 17 can be punctured in the apical region 19, such as in the apical region 19 on the left ventricular wall, to form the opening 50 so as to allow delivery of medical devices and/or therapy to the mitral valve 6.

The apical region 19 is schematically shown in FIG. 1 as the area within the dashed circle. As used herein, the “apical region” can include the inferior tip of the heart 1. The inferior tip is referred to herein as the apex 18 of the heart 1 and is generally located on the midclavicular line, in the fifth intercostal space. The apex 18 can be considered part of the greater apical region 19. Generally, the apical region 19 of the heart is a bottom region of the heart that is within the left or right ventricular region but is distal to the mitral 6 and tricuspid 8 valves and toward the tip of the heart 1. More specifically, the apical region 19 may be considered to be a bottom portion of the heart 1 within about 20 centimeters (cm) to the right or to the left of the median axis of the heart 1.

FIG. 1 shows a tether 20 coupling a leaflet of the mitral valve 6 to the heart wall 17. Mitral valve repair surgeries can comprise deploying one or more tethers onto a mitral valve leaflet for tethering the leaflet to the heart wall 17. Coupling the leaflet to the heart wall 17 can facilitate reshaping of the mitral valve 6, such as to reduce or eliminate leaflet prolapse. The tether 20 can serve to reduce or eliminate leaflet prolapse so as to improve coaptation of the mitral valve 6. In some examples, the tether 20 can be configured to couple the leaflet to a left ventricular portion of the heart wall 17. In some examples, more than one tether can be used to couple the leaflet to the heart wall 17. These tethers can be made from a variety of materials. One or more of these tethers can comprise for example expanded polytetrafluoroethylene (ePTFE). For example, the tether 20 can be an ePTFE suture.

The tether 20 can comprise an elongate portion 22 comprising a proximal portion 24 and a distal portion 26. The proximal portion 24 of the tether 20 can be configured to be coupled to the heart wall 17. The distal portion 26 of the tether 20 can be coupled to the leaflet of the mitral valve 6. In some examples, the distal portion 26 can be associated with an anchor 28 to facilitate securing the tether 20 to the leaflet. The anchor 28 can be positioned at least partially over an upper surface of the leaflet. For example, the anchor 28 can be positioned over an atrial facing surface of the mitral valve leaflet. In some cases, the anchor 28 can comprise a suture knot. In some cases, the suture knot can be formed using the same or similar material as the tether 20. In some cases, the suture knot can be integral with the tether 20. For example, the tether 20 can comprise a suture having a respective portion that forms the anchor 28 and the elongate portion 22. In some cases, the anchor 28 can comprise a bulky-knot anchor.

The proximal portion 24 can be anchored to a portion of the heart wall 17 at or proximate to the apex 18 of the heart 1, such as in the apical region 19. In some examples, a portion of the elongate portion 22 can extend at least partially through the heart wall 17. FIG. 1 shows the tether 20 extending through an entire thickness of the heart wall 17. The proximal portion 24 can be anchored at a position adjacent to an externally facing surface of the pericardium, epicardium or myocardium. In some cases, the elongate portion 22 can comprise a pair of tether tails 30. For example, each of the tether tails 30 can comprise a proximal portion 32 and a distal portion 34. The proximal portions 32 can be anchored to the heart wall 17. Each of the distal portions 34 of the tether tails 30 can be associated with the anchor 28, for example being coupled to the anchor 28. In some cases, the tether tails 30 and the anchor 28 form respective portions of a unitary tether. For example, the tether 20 can comprise a suture having a respective portion that forms the anchor 28 and the tether tails 30.

In some cases, the proximal portion 24 of the tether 20 can be coupled to a surgical pad 40 to facilitate anchoring to the heart wall 17. The tether 20 can couple the mitral valve leaflet to the surgical pad 40, extending from the leaflet through the left ventricle 3 and opening 50 in the heart wall 17 to the surgical pad 40. The surgical pad 40 can be positioned over the opening 50 formed in the heart wall 17. The surgical pad 40 can be over and/or in contact with an externally oriented portion of the heart wall 17. In some cases, the surgical pad 40 can be positioned over and/or adjacent to the pericardium, epicardium or myocardium. For example, the proximal portion 24 of the tether 20 can be coupled to a surgical pad 40 on and in contact with the pericardium, epicardium or myocardium to facilitate anchoring the proximal portion 24 to the target location within the apical region 19 and maintain desired tension in the tether 20. The proximal portion 24 of the tether 20 can be extended through the opening 50 in the heart wall 17, and through the surgical pad 40. In some cases, the tether 20 can be temporarily coupled to the surgical pad 40 to allow adjustment in the tension of the tether 20. The tension of the tether 20 can then be adjusted. As described in further detail herein, the proximal portion 24 can be secured to the surgical pad 40 after a desired tension is achieved in the tether 20. Although FIG. 1 shows one tether 20, it will be understood that more than one tether 20 can be used to anchor the heart valve leaflet to the heart wall 17. It will be understood that tethers 20 can be used to provide tethering for one or more leaflets of a valve other than the mitral valve, such as the tricuspid valve. In some cases, the surgical pad 40 can comprise a pledget.

Adjustment in the tension of the tethers can facilitate providing desired heart valve leaflet coaptation. For example, tension in a tether already coupled to a surgical pad may be fine-tuned after additional tethers have been deployed to the heart valve. As described herein, in some instances, a plurality of tethers can be used. The tethers can be temporarily coupled to the surgical pad to allow subsequent adjustment in the tension of one or more of the tethers. Prior to securing the tethers to the surgical pad, the tension of one or more of the tethers can be adjusted to ensure that the heart valve, such as a mitral valve, can properly coapt. Traditionally, maintaining tension in the tethers can comprise holding onto portions of the tethers extending externally of the opening 50 in the target tissue using clamps, such as rubber-shod clamps. For example, a pair of rubber-shod clamps can be used to clamp onto corresponding portions of the tethers extending externally of the opening in the heart wall to facilitate maintaining desired tension in the tethers. Adjusting the tension of any of the tethers can comprise opening the clamps to facilitate manipulation of the tethers. In some cases, clamping the tethers can result in damage to the tethers, and even breakage of the tethers. While the tensions of the tethers are adjusted, the clamps need to be unclamped such that the tethers are temporarily released. The need to unclamp and release the tethers can limit the accuracy in the tension that is achievable. Tethers can be slippery, thereby impeding manipulation of the tethers and securing of the tethers at desired tensions.

Described herein are systems, devices and methods relating to one or more tether protection devices comprising an inflatable member having an opening extending therethrough. A central portion of the inflatable member can define the opening. The tether protection device can be positioned at least partially over an opening in a target tissue such that the opening of the inflatable member has the same or similar orientation as the opening in the target tissue. One or more tethers extending from the opening in the target tissue can be advanced through the opening of the inflatable member. As described herein, a distal portion of each of the one or more tethers can be coupled to a heart valve leaflet. A proximal portion of each of the one or more tethers can be configured to be coupled to a heart wall to anchor the heart valve leaflet to the heart wall. The inflatable member can be configured to be pressurized to a first inflation state such that at least a portion of the central portion collapses around portions of the one or more tethers disposed within the opening of the inflatable member. For example, at least a portion of the central portion can collapse around proximal portions of the tethers to hold the one or more tethers in place. At least a portion of the central portion can be collapsed around and in contact with the tethers such that the tension of the tethers can be maintained. In some cases, the tether protection device can be configured to be positioned over a surgical pad that is disposed over the opening in the target tissue. Maintaining the inflatable member in the first inflation state can allow an operator, such as a surgeon, to change the position of a tether, such as to adjust the tension of the tether, when the operator is manipulating the tether, while stabilizing the position of the tether when the operator is not manipulating the tether. For example, the operator can adjust the position of the tether and subsequently keep the tether at the adjusted position while confirming coaptation of the heart valve.

The inflatable member of the tether protection device can be pressurized to a second inflation state having a pressure higher than that of the first inflation state to facilitate anchoring the one or more tethers to the target tissue. For example, the one or more tethers can be secured to the surgical pad positioned over the target tissue. The one or more tethers can each be tied to the surgical pad, while the inflatable member is at the second inflation state, after desired tension in each tether is achieved. The increased pressure can facilitate more firmly pinching of the tethers within the opening of the inflatable member, thereby facilitating maintaining the adjusted tensions of the tethers during the tie-down process.

A tether protection device as described herein can improve the accuracy in the tensioning process, while reducing or preventing damage to the tethers. Disposing portions of the tethers within the opening of the inflatable member, such as rather than clamping the tethers, can reduce abrasion to the tethers, thereby eliminating or reducing damage to the tethers. The inflatable member can be configured to hold onto the tethers while the tensions of the tethers are adjusted. For example, the inflatable member can hold onto a tether while a tension of the tether is incrementally adjusted and the coaptation of the heart valve is confirmed, thereby improving the precision of the tension achieved. In some cases, a plurality of tethers can be used. The inflatable member can maintain the tension of other tethers while the position of one tether is adjusted. The tension of each of the tethers can be adjusted without having to release the hold on the other tethers. For example, while the tension for one of the plurality of tethers is adjusted, the central portion can remain collapsed around and in contact with the other tethers such that the tension of the other tethers can be maintained or substantially maintained. Securing the tethers to the surgical pad, such as after desired tension is achieved for each of the tethers, can be performed without having an additional length of the tether due to tying the tethers around a portion of a clamp. Maintaining the hold on the tethers during the tensioning process and securing the tethers without having to tie the tethers to the surgical pad over a clamp can facilitate improved precision in the tension achieved in the tethers. Use of the tether protection devices can facilitate adjustment in the positions of the one or more tethers, including adjustments of up to about 10 millimeters (mm), including up to about 5 millimeters, or up to about 2 millimeters.

In some cases, a pressure of the inflatable member can be adjustable. For example, a level of inflation of the inflatable member can be selected to achieve the desired hold on the one or more tethers. The level of inflation can be selected based on any number of factors, including for example, the number of tethers disposed within the opening of the inflatable member. Adjustability in the pressurization of the inflatable member can facilitate improved grip on the one or more tethers while reducing or preventing damage to the tethers.

The methods, operations, steps, etc. described herein can be performed on a living animal or on a non-living cadaver, cadaver heart, simulator (e.g., with the body parts, tissue, etc. being simulated), etc. For example, methods for treating a patient include methods for simulating the treatment on a simulated patient or anthropogenic ghost, which can include any combination of physical and virtual elements. Examples of physical elements include human or animal cadavers; any portions thereof, including organ systems, whole organs, or tissue; and manufactured elements, which can simulate the appearance, texture, resistance, or other characteristic. Virtual elements can include visual elements provided on a screen, or projected on a surface or volume, including virtual reality and augmented reality implementations. Virtual elements can also simulate other sensory stimuli, including sound, feel, and/or odor.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Although the target tissue is described primarily herein as comprising heart ventricular wall tissue, it will be understood that the target tissue can comprise any number of different types of tissues in the heart and/or in other organs.

In some examples, systems, devices and methods for deploying one or more tethers, as described herein, can comprise one or more features as described in PCT Patent Application No. PCT/US2021/023392, filed Mar. 22, 2021, and entitled “Controlled Suture Tensioning”, which is incorporated herein by reference in its entirety for all purposes.

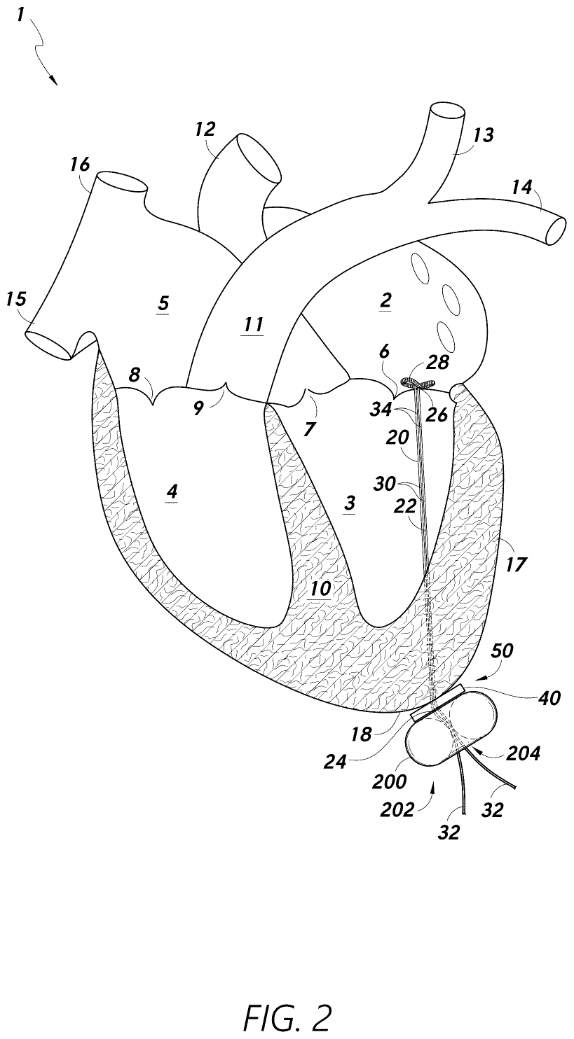

FIG. 2 shows a cross-sectional view of a human heart 1 and a tether 20 deployed to couple a leaflet of a mitral valve 6 to a portion of a heart wall 17 adjacent to the left ventricle 3, where a tether protection device 200 is positioned over an opening 50 formed on the heart wall 17. The tether protection device 200 can comprise an inflatable member 202 and an opening 204 extending therethrough. The tether protection device 200 can be positioned at least partially over an opening in a target tissue. For example, the tether protection device 200 can be positioned at least partially over the opening 50 formed on the heart wall 17 such that the opening 204 of the inflatable member 202 has the same or similar orientation as the opening 50 in the heart wall 17. The proximal portion 24 of the tether 20 can be passed through the opening 204 such that a portion of the tether 20 extending externally of the heart 17 is disposed within the opening 204.

In some cases, the tether protection device 200 can be positioned over the surgical pad 40. For example, the surgical pad 40 can be over and in contact with the heart 1 and the tether protection device 200 can be over and in contact with the surgical pad 40 such that the surgical pad 40 is between the heart 1 and the tether protection device 200. In some cases, the inflatable member 202 can be configured to be maintained at a pressurized state to facilitate tensioning of the tether 20. In some cases, the inflatable member 202 can be configured to be maintained at another pressurized state to facilitate securing of the tether 20 to the surgical pad 40. The inflatable member 202 can be configured to be maintained at a first inflation state such that the opening 204 is occluded or substantially occluded to pinch the portion of the tether 20 disposed within the opening 204 of the inflatable member 202. For example, a diameter of the opening 204 can shrink to zero such that the inflatable member 202 holds onto the tether 20. In some cases, adjusting the tension in the tether 20 can comprise allowing more of the tether 20 to be within the heart 1, such as to reduce the tension in the tether 20, and/or to allow more of the tether 20 to extend externally of the heart 1, such as to increase the tension in the tether 20. The position of the tether 20 can be held by the inflatable member 202 after each adjustment, thereby facilitating verification of the coaptation of the heart valve leaflet. For example, desired coaptation of the target heart valve leaflet of the mitral valve 6 can be confirmed while a portion of the tether 20 is pinched within the opening 204 of the inflatable member 202 such that the position of the tether 20 is maintained. The first inflation state can be configured to allow movement of portions of the tether 20 into and/or out of the opening 50 for adjusting its tension, while maintaining the positions of other tethers that are not being adjusted. The first inflation state can allow an operator, such as a surgeon, to move the tether 20 distally and/or proximally relative to the inflatable member 202 while maintaining the position of the tether 20 while the operator is not manipulating the tether 20.

As described herein, in some cases, a plurality of tethers can be deployed to one or more heart valve leaflets. Portions of the plurality of tethers extending externally of the opening formed in the heart wall can be passed through an opening of an inflatable member. While the tension of one of the plurality of tethers is adjusted, the position of the remaining one or more tethers can be maintained by the inflatable member in the first inflation state. In some cases, maintaining the position of the other tethers while adjusting the tension of one of the tethers can facilitate achieving more accurate tensioning of the plurality of tethers, thereby improving the coaptation of the heart valve. Use of an inflatable member to hold onto the tethers can reduce or eliminate damage to the tethers, for example preventing breakage in the tethers during the tensioning process.

In some cases, the inflatable member 202 can be configured to be pressurized to a second inflation state having a pressure higher than that of the first inflation state to facilitate securing of the tether 20 to the surgical pad 40. After the desired tension is achieved with the tether 20, the tether 20 can be secured to the surgical pad 40. As described herein, the tether 20 can comprise two tether tails 30. The proximal portions 32 of the tether tails 30 can be coupled to one another to secure the tether 20 to the surgical pad 40. For example, the tether tails 30 can be tied together, such as knotted together, to secure the tether 20 to the surgical pad 40. The inflatable member 202 can be maintained at the second inflation state while the tether 20 is secured to the surgical pad 40. The increased pressure of the inflatable member 202 can be configured to more firmly hold the tether 20 at the desired tension while the tether 20 is being tied to the surgical pad 40. For example, the tether tails 30 of the tether 20 can be tied to the surgical pad 40 while the inflatable member 202 is maintained in the second inflation state. The tether 20 can be more stably held in place, avoiding or reducing moving of portions of the tether 20 into and/or out of the heart, while the inflatable member 202 is maintained at the second inflation state. In some cases, a pressure of the inflatable member 202 can be decreased to facilitate removal of the inflatable member 202 from the target site. In some cases, the inflatable member 202 can be depressurized, such as deflated, for removal. In some cases, the inflatable member 202 can be removed after tether 20 is secured to the surgical pad 40. Any number of techniques, other than tying a knot, can be used to fixate the tethers to the surgical pad 40 to maintain the desired tensions (e.g., locking knots and/or SUTRAFIX® clip applier available from Edwards Lifesciences Corp., Irvine, CA).

The inflatable member 202 can be maintained at an initial inflation state while advancing the tether 20 through the opening 204 of the inflatable member 202. The initial pressure can be configured to provide an opening 204 sized to allow advancement therethrough of the tether 20. In some instances, the inflatable member 202 is not collapsed around the tether 20 while in the initial inflation state. The initial inflation state can be a less pressurized state as compared to the first inflation state. For example, a pressure of the inflatable member 202 can be increased from the pressure of the initial inflation state to the pressure of the first inflation state after the tether 20 is positioned through the inflatable member 202. The pressure of the inflatable member 202 can be increased from the first inflation state to the second inflation state after adjustment in the tension of the tether 20 is completed. After the tether 20 is secured to the surgical pad 40, the inflatable member 202 can be depressurized to allow removal of the inflatable member 202. The inflatable member 202 can be depressurized to a depressurized state. In some cases, the depressurized state can be a completely deflated or substantially completely deflated state.

In some cases, more than one tether protection device can be used. For example, a plurality of tether protection devices can be used to facilitate tensioning and/or securing a plurality of tethers. In some cases, a tether protection device can be used for each tether.

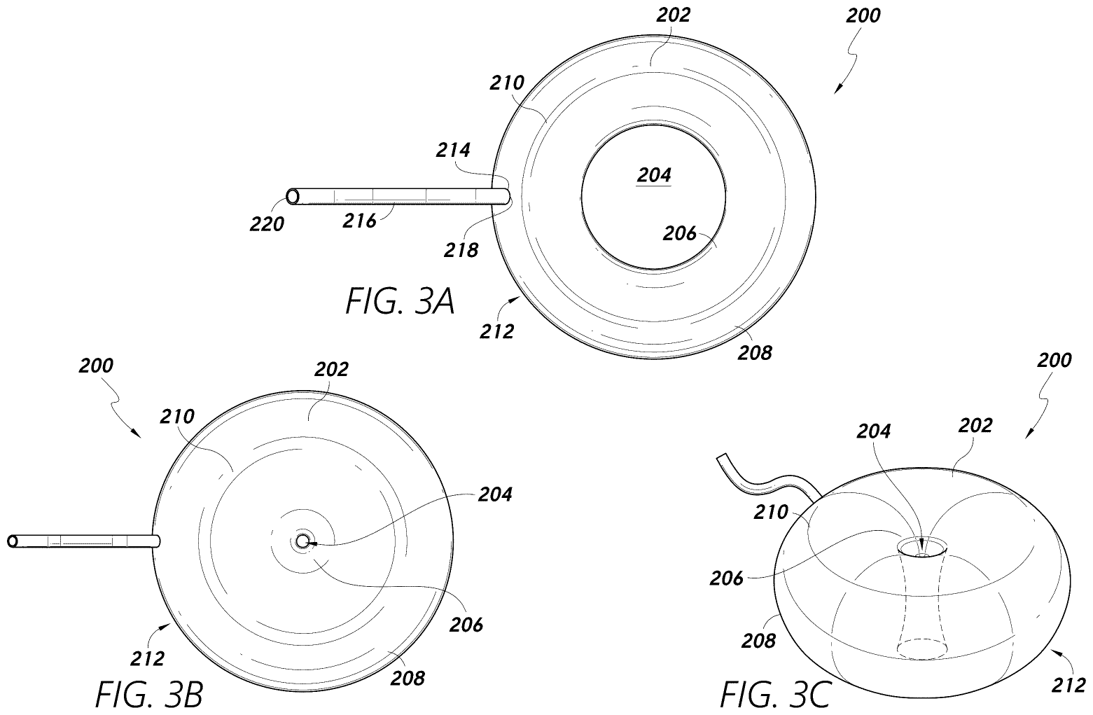

FIGS. 3A, 3B, 3C show various views of the tether protection device 200 described with reference to FIG. 2. FIG. 3A is a top-down view of the tether protection device 200 in a pressurized state and FIG. 3B is a top-down view of the tether protection device 200 in another pressurized state that is more pressurized than that shown in FIG. 3A. FIG. 3C is a perspective view of the tether protection device 200 in the state shown in FIG. 3B.

Referring to FIG. 3A, the tether protection device 200 can comprise an inflatable member 202. An opening 204 can extend through the inflatable member 202 and be configured to receive a portion of one or more tethers. As described herein, the tether protection device 200 can be positioned at least partially over an opening in a target tissue such that the opening 204 of the inflatable member 202 has the same or similar orientation as the opening in the target tissue. An upper portion 210 of the inflatable member 202 can be oriented away from the target tissue and a lower portion 212 can be oriented toward the target tissue. For example, the lower portion 212 can be positioned over and/or in contact with a surgical pad positioned over the opening in the target tissue. At least one tether extending from the opening in the target tissue can be positioned through the opening 204. For example, a plurality of tethers can each comprise a corresponding portion disposed within the opening 204 of the inflatable member 202. A central portion 206 of the inflatable member 202 can define the opening 204.

The tether protection device 200 can comprise a fluid port 214 associated with the inflatable member 202. Fluid can be introduced into an internal volume of the inflatable member 202 to inflate the inflatable member 202 and/or be withdrawn from the internal volume of the inflatable member 202 to deflate the inflatable member 202, through the fluid port 214. In some examples, the fluid port 214 can comprise an opening extending through a wall portion of the inflatable member 202 such that fluid can be injected and withdrawn from the internal volume of the inflatable member 202. The fluid port 214 can be configured to mate with an injection device configured to provide the fluid. In some cases, the injection device can comprise a syringe. The fluid can be a liquid or a gas. In some examples, the fluid can be a saline solution. In some examples, a fluid tube member 216 can be associated with the fluid port 214 to provide a lumen configured to facilitate delivery and/or withdrawal of the fluid from the inflatable member 202. A first end 218 of the fluid tube member 216 can be configured to be coupled to the fluid port 214 and a second end 220 of the fluid tube member 216 can be configured to be coupled to a fluid injection device.

The inflatable member 202 can be pressurized to reduce a size of its opening 204. The tether protection device 200 shown in FIGS. 3B and 3C comprises the inflatable member 202 in a more pressurized state than that shown in FIG. 3A. In some instances, either of the inflation states shown in FIGS. 3A and 3B can be an initial inflation state. As fluid is introduced into the inflatable member 202, the inflatable member 202 can be configured to expand such that the central portion 206 extends inward toward a longitudinal axis of the inflatable member 202 to reduce a diameter of the opening 204 defined by the central portion 206. The longitudinal axis of the inflatable member 202 can extend between the upper portion 210 and the lower portion 212. In some cases, the longitudinal axis can be perpendicular or substantially perpendicular to an orientation of the opening 204. As shown, the opening 204 in FIG. 3B is smaller in size than that shown in FIG. 3A.

The plurality of tethers can be advanced through the opening 204 such that corresponding portions of the tethers are disposed within the opening 204. In some examples, the inflatable member 202 can be configured to be maintained at a pressure such that the size of the opening 204 allows the tethers to move freely within the opening 204. In some examples, the tether protection device 200 as shown in FIGS. 3A and 3B can be configured to allow the plurality of tethers to move freely within the opening 204.

In some examples, the inflatable member 202 can be configured to be maintained at an initial inflation state where the opening 204 is sized to allow the plurality of tethers to move freely therewithin. For example, the pressurized state as described with reference to FIG. 3A or 3B can be an initial inflation state. In the initial inflation state, the plurality of tethers can move freely within the opening 204. In some examples, the opening 204 can have a circular or substantially circular shape while the inflatable member 202 is in the first inflation state. For example, while in the initial inflation state, the central portion 206 of the inflatable member 202 can define a circular or substantially circular opening extending through the inflatable member 202. In some examples, a circumferential portion 208 of the inflatable member 202 can form a circular or substantially circular shape. For example, the inflatable member 202 can have a circular or substantially circular ring shape. The inflatable member 202 can have a circular doughnut shape.

In some examples, the upper portion 210 and/or the lower portion 212 can be curved. A profile of the upper portion 210 and/or lower portion 212 along a plane containing the longitudinal axis of the inflatable member 202 can comprise a convex curve. In some examples, while the inflatable member 202 is in the initial inflation state, the profile of the upper portion 210 and/or lower portion 212 can comprise a segment of a circle. For example, the inflatable member 202 can have a torus shape while the inflatable member 202 is in the initial inflation state.

In some examples, the inflatable member 202 can be configured to be maintained at a pressure such that at least a portion of the central portion 206 pinches the plurality of tethers. For example, the inflatable member 202 can be configured to be maintained at a pressure such that at least a portion of the central portion 206 collapses around the plurality of tethers extending through the opening 204. The inflatable member 202 can be inflated to a pressurized state such that the central portion 206 makes sufficient contact with corresponding portions of the plurality of tethers to reduce or prevent movement of the tethers. For example, the inflatable member 202 can be inflated to a first inflation state that is more pressurized than the initial inflation state such that contact between the corresponding portions of the central portion 206 and plurality of tethers can be maintained or substantially maintained. The inflatable member 202 can be maintained at the first inflation state to facilitate adjusting the tension of at least one of the plurality of tethers. The first inflation state can be configured to allow both movement of the tether through the opening 204 while an operator, such as a surgeon, is manipulating the tether, and to reduce or prevent movement of the tether while the operator is not manipulating the tether. For example, while the inflatable member 202 is maintained in the first inflation state, a tension of a tether can be adjusted. A portion of the tether can be move into or out of the the heart ventricle to achieve the desired tension. The tether can then be held at the desired tension by maintaining the desired portion of the tether within the opening 204 while the inflatable member 202 is maintained in the first inflation state. The central portion 206 can maintain contact with a corresponding portion of the tether to provide the desired tension in the tether. In some examples, while the tether protection device 200 is in the first inflation state, the central portion 206 can be extended and/or disposed inward toward the longitudinal axis such that the opening 204 is occluded or substantially occluded. For example, the tether protection device 200 can be inflated until the central portion 206 extends and/or is disposed inward toward the longitudinal axis such that the opening 204 is occluded or substantially occluded. In some examples, a diameter of the opening 204 in the first inflation state can be zero or substantially zero.

In some examples, the tether protection device 200 can be configured to be inflated to a second inflation state that is more pressurized than the first inflation state. The tether protection device 200 can be maintained at the second inflation state to facilitate securing at least one of the plurality of tethers at a desired tension. For example, a tether, including a pair of tether tails of a tether, can be secured to the surgical pad to maintain the tether or pair of tether tails at a desired tension. In some examples, a knot can be formed using the tether, or pair of tether tails of the tether, to secure the tether or pair of tether tails. In some examples, while the tether protection device 200 is in the second inflation state, the central portion 206 can be extended and/or disposed inward toward the longitudinal axis such that the opening 204 is occluded or substantially occluded. For example, the tether protection device 200 can be inflated from the first inflation state to the second inflation state such that the central portion 206 presses harder and/or further inward toward the longitudinal axis within the opening 204, as compared to that in the first inflation state, to make stronger frictional contact with corresponding portions of the plurality of tethers. In some examples, a diameter of the opening 204 in the second inflation state can be zero or substantially zero.

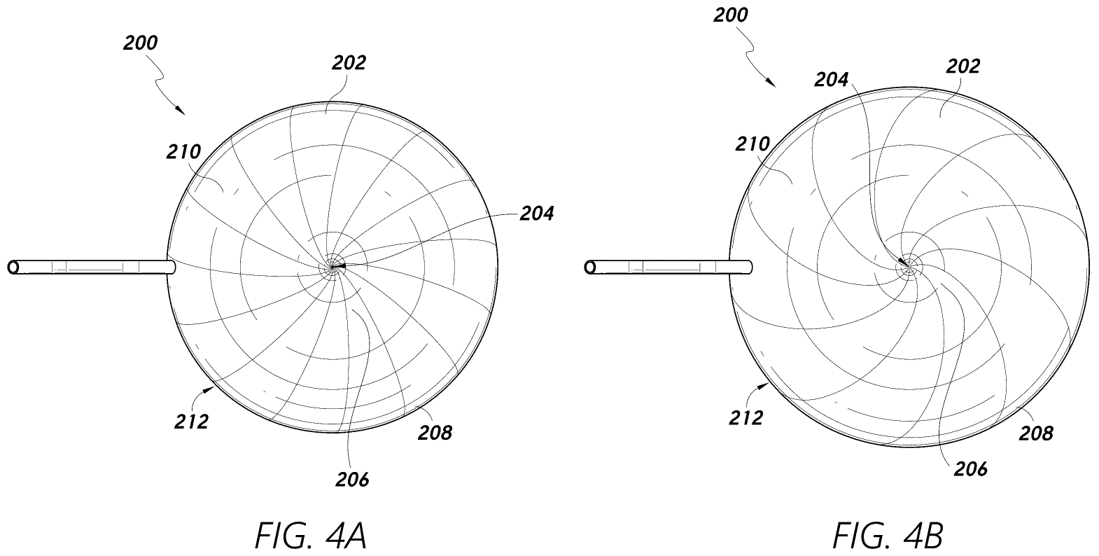

FIGS. 4A and 4B show the tether protection device 200 in various pressurized states. The tether protection device 200 shown in FIG. 4B is more pressurized than that shown in FIG. 4A. In some examples, FIG. 4A shows the inflatable member 202 in a first inflation state and FIG. 4B shows the inflatable member 202 in a second inflation state. In the first and second inflation states, the central portion 206 of the inflatable member 202 can extend and/or be disposed inward toward the longitudinal axis such that the opening 204 of the inflatable member 202 is occluded or substantially occluded. The central portion 206 of the inflatable member 202 can press harder against itself while the inflatable member 202 is in the second inflation state than in the first inflation state, such that the central portion 206 exerts increased frictional contact with the plurality of tethers.

The inflatable member 202 can be configured to be maintained at the pressures described herein. For example, the circumferential portion 208 can be configured to maintain the desired configuration, such as in a circular shape, at the first inflation state and the second inflation state, while the central portion 206 is configured to be collapsed around and in contact with the plurality of tethers. In some cases, the pressure of the inflatable 202 can be adjustable, such as to accommodate more or fewer tethers. The inflatable member 202 can comprise any number of materials, including compliant materials. For example, the central portion 206 of the inflatable member 202 can comprise a compliant material configured to facilitate collapsing at least a portion of the central portion 206 around the tethers. In some cases, the inflatable member 202 can comprise latex. For example, the inflatable member 202 can be a latex inflatable member having a torus shape.

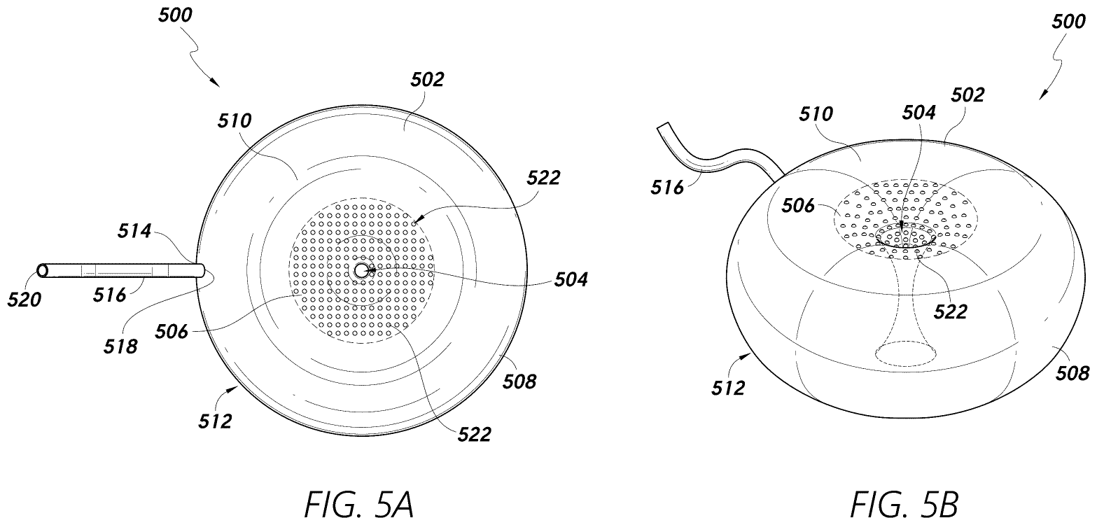

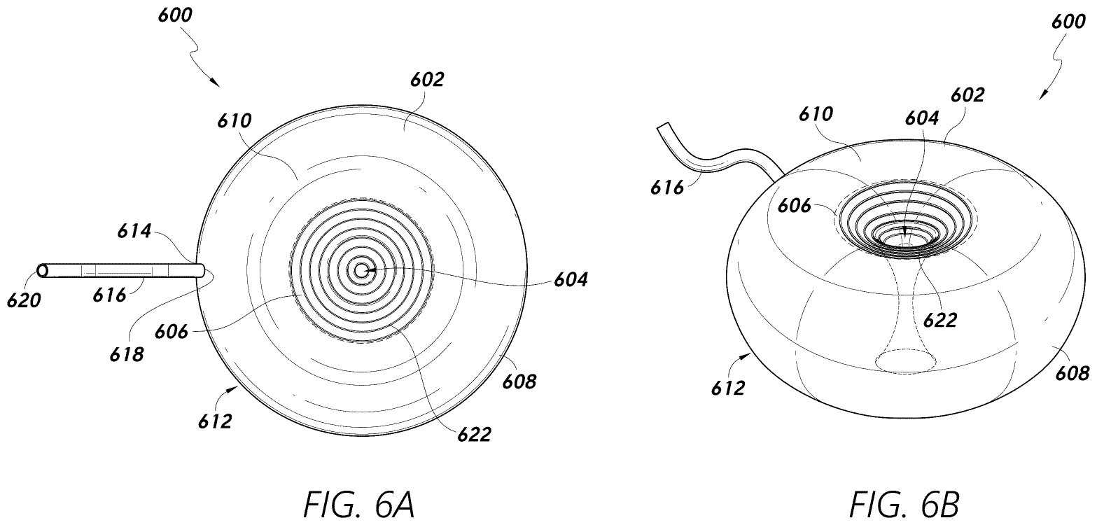

In some examples, one or more externally oriented surface portions of an inflatable member can comprise a texture to facilitate increased frictional contact between the inflatable member and corresponding portions of the plurality of tethers. The texture can provide additional surface area to contact the corresponding portions of the plurality of tethers. FIGS. 5 and 6 show examples tether protection devices comprising textured inflatable members. FIG. 5A is a top-down view, and FIG. 5B is a perspective view, of an example of a tether protection device 500 comprising an inflatable member 502 having a plurality of bumps 522 thereon. A central portion 506 of the inflatable member 502 defining an opening 504 extending through the inflatable member 502 can comprise the plurality of bumps 522. The plurality of bumps 522 can contact the plurality of tethers while at least a portion of the central portion 506 is collapsed around the plurality of tethers, such as while the inflatable member 502 is in a first inflation and a second inflation state.

In some examples, another portion of the inflatable member 502 can comprise the plurality of bumps 522. An upper portion 510 and/or a lower portion 512 of the inflatable member 502 can comprise the bumps 522. For example, both an upper and lower portion of the central portion 506 of the inflatable member 502 can comprise the bumps 522.

FIG. 6A is a top-down view, and FIG. 6B is a perspective view, of an example of a tether protection device 600 comprising an inflatable member 602 having a plurality of ridges 622. In some examples, the ridges 622 can be oriented perpendicularly or substantially perpendicularly relative to a longitudinal axis of the inflatable member 602. The longitudinal axis of the inflatable member 602 can extending between an upper portion 61o and a lower portion 612 of the inflatable member 602. In some examples, the inflatable member 602 can comprise a ring shape, including a circular or substantially circular ring shape, such as while the inflatable member 602 is in an inflated state. In some examples, the inflatable member 602 can have a torus shape. The plurality of ridges 622 can have a circular or substantially circular shape, such as extending and/or being disposed circumferentially around a respective portion of the ring-shaped inflatable member 602.

In some examples, the plurality of ridges 622 can be on a central portion 606 of the inflatable member 602, including a portion of the inflatable member 602 defining an opening 604 extending through the inflatable member 602. In some examples, another portion of the inflatable member 602 can comprise the plurality of ridges 622. The upper portion 61o and/or a lower portion 612 of the inflatable member 602 can comprise the ridges 622. For example, an upper and/or a lower portion of the central portion 606 can comprise the ridges 622.

The plurality of bumps 522 and the plurality of ridges 622 can provide additional frictional contact between the inflatable member 502, 602 and the respective plurality of tethers. A tether protection device can comprise one or more other types of textures. The texture on a surface portion of the inflatable member can provide added surface contact between corresponding portions of the plurality of tethers and the inflatable member without interfering with maintaining the tethers within the opening. For example, the added surface contact between the tethers and the tether protection device can provide desired friction between the two to securely maintain corresponding portions of the plurality of tethers disposed within the opening of the tether protection device, without being too large as to interfere with holding onto the plurality of tethers.

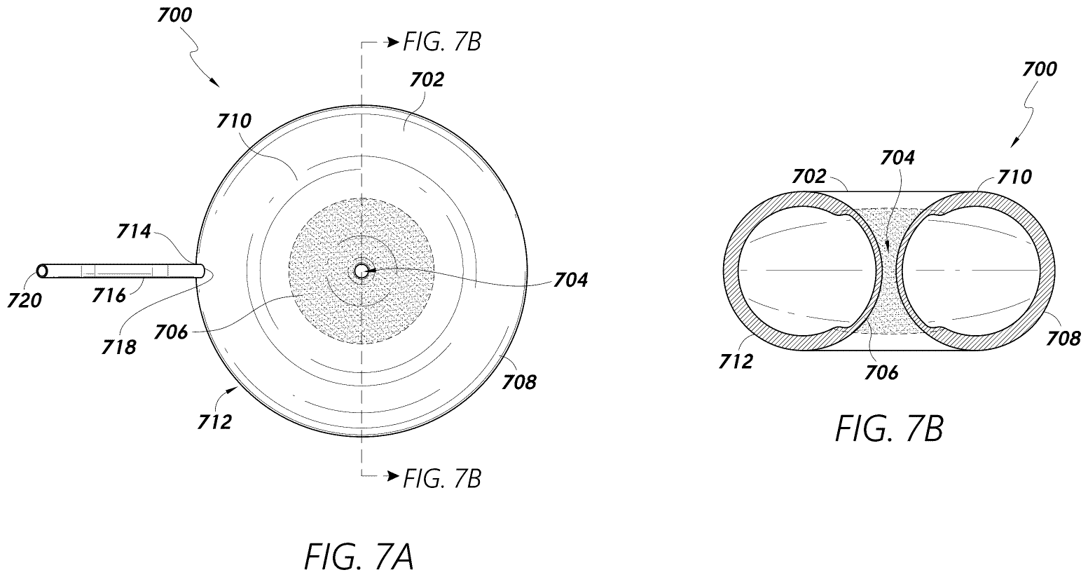

In some examples, an inflatable member of a tether protection device can comprise portions having different wall thicknesses. One or more portions of an inflatable member can comprise a wall thickness thinner than one or more other portions of the device. FIG. 7A is a top-down view, and FIG. 7B is a cross-sectional view, of an example of a tether protection device 700 comprising an inflatable member 702 having a central portion 706 that has a wall thickness less than a wall thickness of a circumferential portion 708 of the inflatable member 702. As shown in FIG. 7A, the central portion 706 defines an opening 704 extending through the inflatable member 702. One or more portions of the central portion 706 can comprise a wall thickness thinner than one or more other portions of the inflatable member 702. A thinner wall thickness for the central portion 706 can facilitate collapsing at least a portion of the central portion 706 around the plurality of tethers. In some examples, a circumferential portion 708 of the inflatable member 702 can have a wall thickness thicker than that of one or more portions of the central portion 706. For example, the central portion 706 can have a wall thickness thinner than that of the circumferential portion 708. In some examples, the central portion 706 having a thinner wall thickness than the circumferential portion 708 to facilitate collapsing of the central portion 706 around the plurality of tethers when the inflatable member 702 is pressurized. The thicker wall thickness of the circumferential portion 708 can facilitate maintaining the circumferential portion 708 in the desired configuration, such as in a circular or substantially circular configuration, while the inflatable member 702 is in various inflation states. FIGS. 7A and 7B show the circumferential portion 708 maintaining the circular or substantially circular configuration while the inflatable member 702 is in an inflation state. An upper portion 710 of the inflatable member 702 can be configured to be oriented away from an opening formed in a target tissue. A lower portion 712 of the inflatable member 702 can be configured to be oriented toward the opening formed in the target tissue.

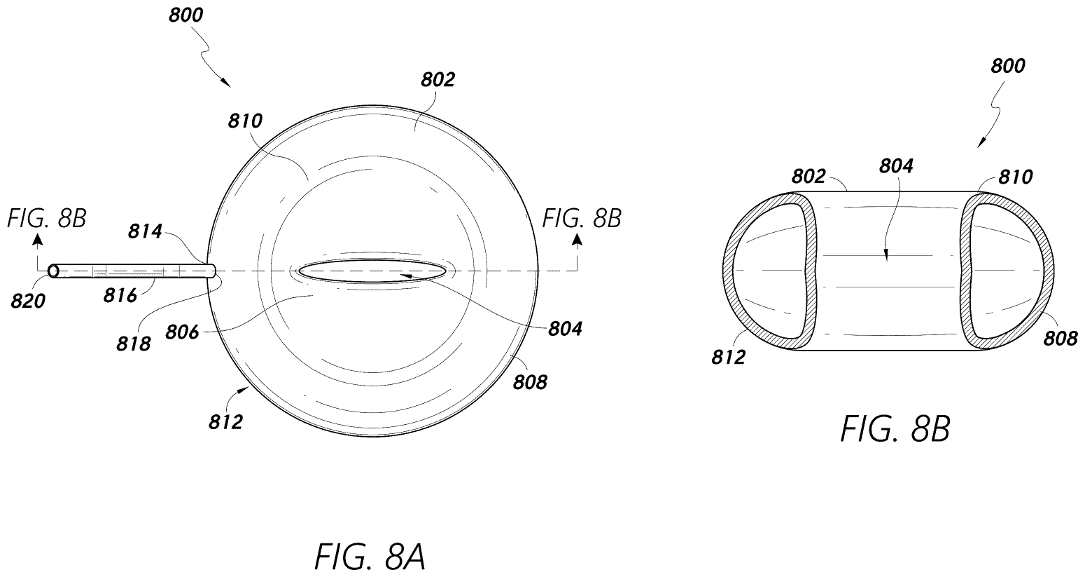

An inflatable member of a tether protection device can comprise an opening having a shape other than a circle. FIG. 8A is a top-down view, and FIG. 8B is a cross-sectional view taken along the plane shown in FIG. 8A, of an example of a tether protection device 800 comprising an inflatable member 802 that has an elongate shaped opening 804. As shown in FIGS. 8A and 8B, a central portion 806 of the inflatable member 802 can define an opening 804 having an elongate shape. A circumferential portion 808 of the inflatable member 802 can be configured to maintain a circular or substantially circular configuration while the inflatable member 802 is in various inflation states, such as the inflation state shown in FIGS. 8A and 8B. An upper portion 81o of the inflatable member 802 can be configured to be oriented away from an opening formed in a target tissue. A lower portion 812 of the inflatable member 802 can be configured to be oriented toward the opening formed in the target tissue. For example, the opening 804 have an elongate shape while the inflatable member 802 is in an initial inflation state as described herein. The inflatable member 802 can be inflated to a first inflation state and/or a second inflation state as described herein such that space within the opening 804 disappears and at least a portion of the central portion 806 collapses around a plurality of tethers extending through the opening 804. In some cases, the elongate shape of the opening 804 can facilitate providing a distance between the plurality of tethers disposed through the opening 804 so as to distinguish between the various tethers.

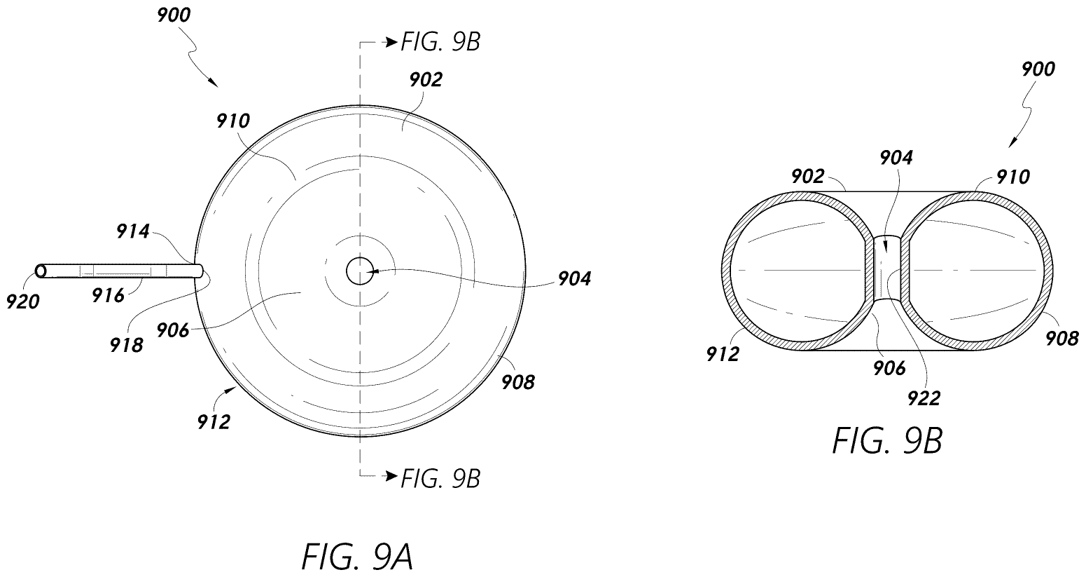

FIGS. 9A and 9B show an example of a tether protection device 900 comprising an inflatable member 902 that has a tubular lumen 904 extending therethrough. FIG. 9A is a top-down view, and FIG. 9B is a cross-sectional view taken along the plane shown in FIG. 9A, of the tether protection device 900. A central portion 906 of the inflatable member 902 can define the tubular lumen 904. A plurality of tethers can be configured to be advanced through the tubular lumen 904. At least a portion of the inflatable member 902 defining the tubular lumen 904 can be configured to collapse around and contact portions of the plurality of tethers disposed within the tubular lumen 904 to hold the plurality of tethers in place. The tubular configuration can facilitate improved contact between the inflatable member 902 and the plurality of tethers. For example, the tubular configuration can provide increased surface area to contact portions of the plurality of tethers disposed within the tubular lumen 904.

The tether protection device 900 can be positioned over a target tissue, including an opening formed in the target tissue, such that an upper portion 910 of the inflatable member 902 can be configured to be oriented away from the target tissue and a lower portion 912 of the inflatable member 902 can be configured to be oriented toward the target tissue. The tubular lumen 904 can be configured to be perpendicularly oriented relative to the target tissue while the inflatable member 902 is positioned over the target tissue. The tubular lumen 904 can be coaxial with a longitudinal axis of the inflatable member 902. The longitudinal axis of the inflatable member 902 can extend between an upper portion 910 and a lower portion 912 of the inflatable member 902.

FIGS. 9A and 9B show the inflatable member 902 in an initial inflation state. A circumferential portion 908 of the inflatable member 902 can be configured to maintain a circular or substantially circular configuration while the inflatable member 902 is in various inflation states, including the initial inflation state. For example, the plurality of tethers can be configured to be allowed to move freely within the tubular lumen 904 in the initial inflation state. The tubular lumen 904 can have a cylindrical shape while the inflatable member 902 is in the initial inflation state. For example, a lumen surface portion 922 can define the tubular lumen 904. The lumen surface portion 922 can define a cylindrical tubular lumen. A cross-section the inflatable member 902 along a plane perpendicular to the longitudinal axis of the inflatable member 902 can comprise a circle. For example, an opening forming the cross-section of the tubular lumen 904 can form a circular or substantially circular shape. In some cases, a tubular lumen can have a shape other than a cylinder. In some cases, a tubular lumen can have a cross-section which forms an elongate shape.

The inflatable member 902 can be inflated to a first inflation state and/or a second inflation state such that at least a portion of the lumen surface portion 922 collapses around the corresponding portions of the plurality of tethers disposed within the tubular lumen 904. In some cases, the entire portion of the lumen surface portion 922 can collapse around the tethers to facilitate improved contact between the inflatable member 902 and the tethers.

The tether protection devices 500, 600, 700, 800, 900 can have one or more other features described with reference to the tether protection device 200 described with reference to FIGS. 2 through 4. For example, each of the tether protection devices 500, 600, 700, 800, 900 can comprise a fluid port 514, 614, 714, 814, 914 associated with the corresponding inflatable member 502, 602, 702, 802, 902. In some examples, the fluid ports 514, 614, 714, 814, 914 can each comprise an opening extending through a wall portion of the corresponding inflatable members 502, 602, 702, 802, 902. Fluid can be injected into each of the inflatable members 502, 602, 702, 802, 902 so as to inflate the corresponding inflatable member 502, 602, 702, 802, 902 to a first inflation state or a second inflation state. Fluid can be withdrawn from each of the inflatable members 502, 602, 702, 802, 902 to depressurize the inflatable members 502, 602, 702, 802, 902. A fluid tube member 516, 616, 716, 816, 916 can be associated with each of the fluid ports 514, 614, 714, 814, 914 to provide a lumen configured to facilitate delivery and/or withdrawal of the fluid from the inflatable members 502, 602, 702, 802, 902. A first end 518, 618, 718, 818, 918 of each of the fluid tube members 516, 616, 716, 816, 916 can be configured to be coupled to the respective fluid port 514, 614, 714, 814, 914 and a second end 520, 620, 720, 820, 920 can be configured to be coupled to a fluid injection device.

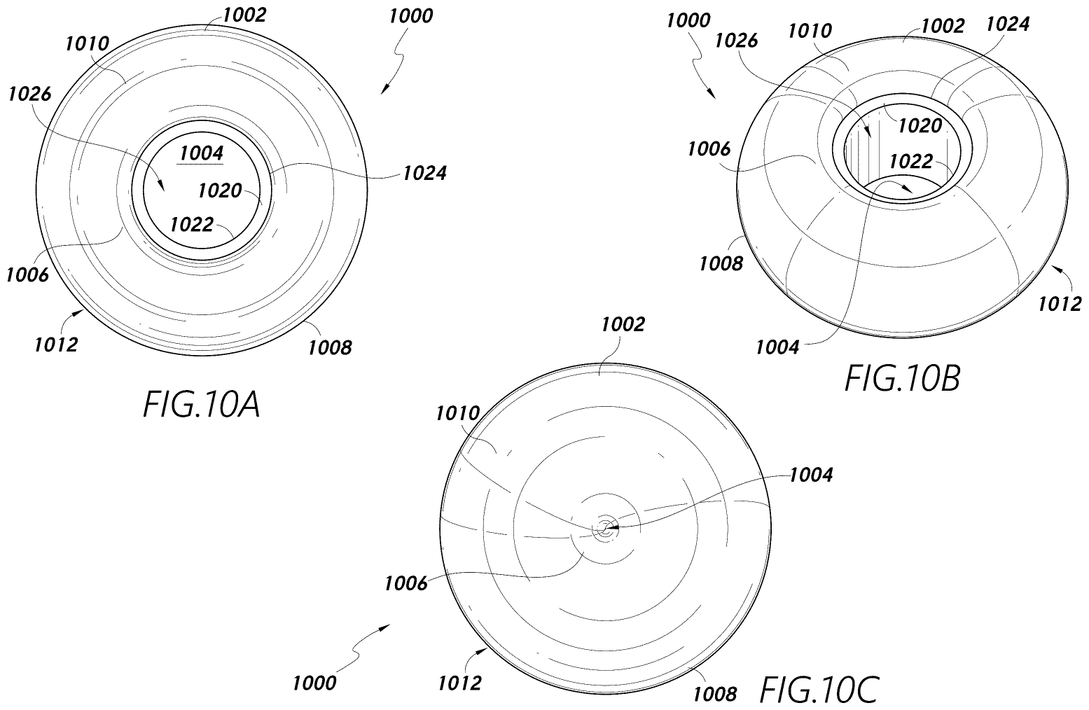

FIGS. 10A, 10B and 10C show various views of an example of a tether protection device 1000 comprising an inflatable member 1002 and a ring member 1020 configured to be disposed within an opening 1004 of the inflatable member 1002. A central portion 1006 of the inflatable member 1002 can define the opening 1004. FIG. 10A is a top-down view, and FIG. 10B is a perspective view, of the tether protection device 1000 in a first configuration comprising the ring member 1020 disposed within the opening 1004 of the inflatable member 1002. FIG. 10C is a top-down view of the tether protection device 1000 in a second configuration where the ring member 1020 is removed from the inflatable member 1002. The ring member 1020 can be configured to be positioned within the opening 1004 to facilitate advancement therethrough of a plurality of tethers. The ring member 1020 can be subsequently removed after desired portions of the plurality of tethers are disposed within the opening 1004 of the inflatable member 1002.

Referring to FIGS. 10A and 10B, the inflatable member 1002 can be in an inflation state configured to facilitate maintaining the plurality of tethers at a desired tension. For example, the inflatable member 1002 can be in a first inflation state as described herein such that at least a portion of the central portion 1006 collapses inward toward a longitudinal axis of the inflatable member once the ring member 1020 is removed. The longitudinal axis can extend between an upper portion 1010 and a lower portion 1012 of the inflatable member 1002. The upper portion 1010 can be configured to be oriented away from a target tissue, including an opening formed in the target tissue. The lower portion 1012 can be configured to be oriented toward the target tissue. The central portion 1006 can collapse around and contact corresponding portions of the plurality of tethers disposed within the opening 1004 so as to keep each of the plurality of tethers at a respective tension. FIGS. 10A and 10B show the ring member 1020 disposed within the opening 1004 to maintain an aperture extending through the inflatable member 1002. The ring member 1020 can be configured to be positioned within the opening 1004 extending through the inflatable member 1002 such that the opening 1026 extending through the ring member 1020 has the same or similar orientation as the opening 1004 of the inflatable member 1002. A first inner surface portion 1022 of the ring member 1020 defining the opening 1026 can oriented toward the longitudinal axis of the inflatable member 1002. A second outer surface portion 1024 of the ring member 1020 can be oriented toward and contact the central portion 1006 that defines the opening 1004 of the inflatable member 1002. The ring member 1020 can be kept in the opening 1004 of the inflatable member 1002 to allow the plurality of tethers to be advanced therethrough.

Referring to FIG. 10C, in the second configuration, the ring member 1020 is not disposed within the opening 1004 of the inflatable member 1002. The ring member 1020 can be removed after the plurality of tethers is threaded through the opening 1004. A relaxed state of the inflatable member 1002 can comprise at least a portion of the central portion 1006 collapsed inward toward the longitudinal axis, such that once the ring member 1020 is removed, the central portion 1006 assumes the collapsed state. For example, the relaxed state of the inflatable member 1002 can be the first inflation state.

The tether protection device 1000 can have one or more other features described with reference to the tether protection device 200 described with reference to FIGS. 2 through 4. For example, the tether protection 1000 can comprise a fluid port associated with the inflatable member 1002. A fluid tube member can be associated with the fluid port to provide a lumen configured to facilitate delivery and/or withdrawal of the fluid from the inflatable member. A circumferential portion 1008 of the inflatable member 1002 can be configured to maintain a circular or substantially circular configuration while the inflatable member 1002 is in various inflation states, such as the inflation state shown in FIGS. 10A, 10B and 10C.

In some cases, the inflatable member 1002 can be pressurized after tensions in the plurality of tethers are adjusted to facilitate securing the tethers to a surgical pad. The inflatable member 1002 can be inflated from the first inflation state to a second inflation state having a pressure higher than that of the first inflation state.

In some cases, a tether protection device can comprise an inflatable member configured to be in a first inflation state in its relaxed state. The opening of the inflatable member can be maintained to allow positioning therethrough of a plurality of tethers. The central portion of the inflatable member defining the opening can be allowed to collapse around the plurality of tethers to hold onto the tethers while the respective tensions are adjusted. In some cases, allowing the central portion to collapse can comprise pulling a vacuum. For example, pressure maintaining the opening can be released to allow the collapse of the central portion.

In some examples, a suture system can comprise a plurality of tether protection devices. The plurality of tether protection devices can have features as described herein. For example, the suture system can comprise a plurality of tethers and a plurality of tether protection devices. The plurality of tethers can be configured to extend from an opening in a target tissue. Each of the plurality of tethers can comprise a first portion configured to extend into the opening in the target tissue and a second portion configured to extend externally from the opening in the target tissue. Each of the plurality of inflatable members can be configured to be positioned over the opening in the target tissue such that an opening of each of the plurality of inflatable members can be configured to have an orientation similar or the same as that of the opening in the target tissue. A corresponding portion of one or more tethers can be configured to be disposed through an opening of one of the tether protection devices and a corresponding portion of one or more other tethers can be configured to be disposed through an opening of another of the tether protection devices. In some examples, a tether protection device can be used for each pair of the plurality of tethers. For example, one pair of tether tails can be disposed within an opening of one inflatable member.

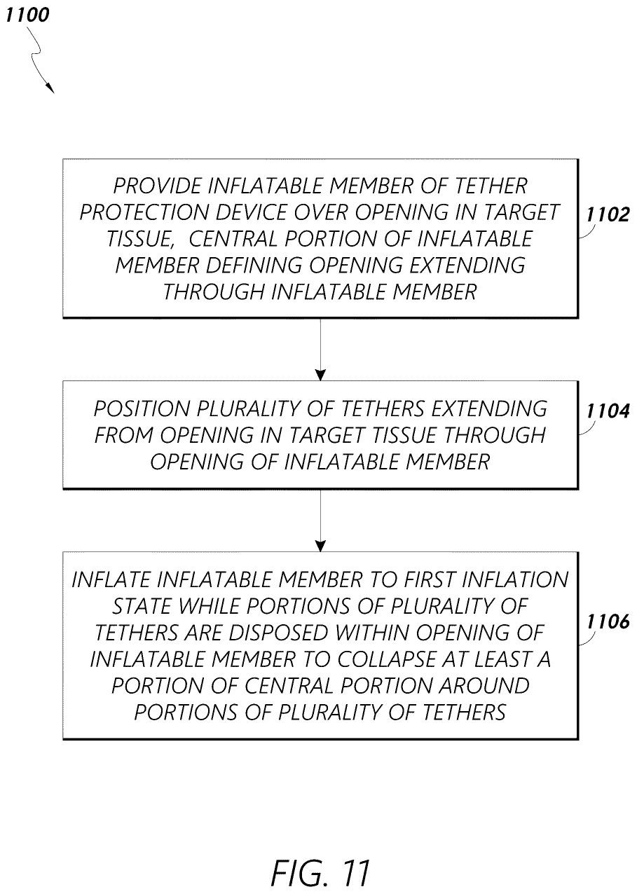

FIG. 11 is a flow diagram of an example of a process 1100 for tensioning a plurality of tethers. One or more tether protection devices as described herein can be used. In block 1102, the process can involve providing a tether protection device over an opening in a target tissue. The tether protection device can have one or more features as described herein. For example, the tether protection device can comprise an inflatable member that has a central portion defining an opening extending through the inflatable member. While the inflatable member is positioned over the target tissue, the inflatable member can be oriented such that the opening of the inflatable member has the same orientation as the opening in the target tissue.

In block 1104, the process can involve positioning a plurality of tethers extending from the opening in the target tissue through the opening of the inflatable member. Corresponding portions of the plurality of tethers can be disposed within the opening of the inflatable member. In some cases, the inflatable member can be in an initial inflation state while the plurality of tethers is positioned through the opening of the inflatable member. In the initial inflation state, the plurality of tethers can be allowed to move freely within the opening of the inflatable member.

In block 1106, the process can involve collapsing at least a portion of the central portion around respective portions of the plurality of tethers while the portions of the plurality of tethers are disposed within the opening of the inflatable member. The respective positions of the plurality of tethers can be maintained while at least a portion of the central portion of the inflatable member is collapsed around and in contact with the plurality of tethers.

In some cases, collapsing at least a portion of the central portion can comprise pressurizing the inflatable member to a first inflation state. For example, the inflatable member can be pressurized from the initial inflation state to the first inflation state such that at least a portion of the central portion collapses around respective portions of the plurality of tethers disposed within the opening of the inflatable member and holds onto the tethers.

In some examples, the process can involve adjusting the tension of the plurality of tethers while the plurality of tethers is disposed within the opening of the inflatable member and while at least a portion of the central portion is collapsed around respective portions of the plurality of tethers. A tension of one of the tethers can be adjusted while the tension of the other tethers can be maintained

As described herein, a surgical pad can be positioned at least partially over the opening in the target tissue. The plurality of tethers can be secured to the surgical pad, such as after desired tension in each of the tethers is achieved. In some examples, the process can involve inflating the inflatable member to a second inflation state, the second inflation state being more pressurized than the first inflation state. The inflatable member can be inflated to the second inflation state to facilitate securing the plurality of tethers to the surgical pad. For example, a knot can be formed using a pair of tether tails of the tether to tie the tether to the surgical pad. The second inflation state can reduce or eliminate movement of any portion of each of the plurality of tethers into and/or out of the opening in the target tissue so as to maintain the adjusted tension in the tethers. The inflatable member can be maintained at the second inflation state during the tie-down process.

In some examples, providing the tether protection device can comprise providing the inflatable member in the first inflation state and a ring positioned within the opening of the inflatable member. The plurality of tethers can be positioned through the opening of the inflatable member while the ring is positioned within the opening. The ring can be removed from the opening of the inflatable member to allow at least a portion of the central portion of the inflatable member to collapse around the plurality of tethers. The tension of one or more of the plurality of tensions can be adjusted while the central portion of the inflatable member is collapsed around the plurality of tethers.

### Additional Examples

Example 1: A tether protection device, comprising:
an inflatable member configured to be positioned over an opening in a target tissue, the inflatable member comprising a central portion defining an opening extending through the inflatable member, the opening of the inflatable member being configured to allow for a plurality of tethers extending from the opening in the target tissue to be disposed at least partially therein; and
a fluid port associated with the inflatable member, the inflatable member being configured to be inflated via the fluid port to:
an initial inflation state in which the plurality of tethers is allowed to move freely within the opening of the inflatable member, and
a first inflation state in which at least a portion of the central portion is collapsed around and in contact with the plurality of tethers.

Example 2: The device of any of the examples described herein, in particular example 1, wherein an externally oriented surface of the central portion comprises a texture.

Example 3: The device of any of the examples described herein, in particular example 2, wherein the texture comprises at least one of a plurality of bumps and a plurality of ridges.

Example 4: The device of any of the examples described herein, in particular examples 1 to 3, wherein a wall thickness of the central portion is less than a wall thickness of a circumferential portion of the inflatable member.

Example 5: The device of any of the examples described herein, in particular examples 1 to 4, wherein a circumferential portion of the inflatable member forms a circular shape.

Example 6: The device of any of the examples described herein, in particular examples 1 to 5, wherein the central portion defines a tubular lumen configured to be perpendicularly oriented relative to the target tissue while the inflatable member is positioned over the target tissue, the opening of the inflatable member being a cross-sectional portion of the tubular lumen.

Example 7: The device any of the examples described herein, in particular example 6, wherein the tubular lumen forms a cylindrical shape.

Example 8: The device of any of the examples described herein, in particular examples 1 to 7, wherein the opening of the inflatable member comprises a circular shape while the inflatable member is in the first inflation state.

Example 9: The device of any of the examples described herein, in particular examples 1 to 6, wherein the opening of the inflatable member comprises an elongate shape while the inflatable member is in the first inflation state.

Example 10: The device of any of the examples described herein, in particular examples 1 to 9, wherein the inflatable member is configured to be maintained in a second inflation state while a pair of tether tails of the plurality of tethers is tied together, the second inflation state being more pressurized than the first inflation state.

Example 11: The device of any of the examples described herein, in particular examples 1 to 10, wherein the inflatable member is configured to be in the first inflation state and wherein the device further comprises a ring configured to be positioned in the opening of the inflatable member to maintain the opening and allow advancement therethrough of the plurality of tethers.

Example 12: A suture system, comprising:
a plurality of inflatable members configured to be positioned over an opening in a target tissue, each of the plurality of inflatable members comprising:
a central portion defining an opening extending through the inflatable member, the opening of the inflatable member being configured to have the same orientation as the opening in the target tissue while the inflatable member is positioned over the target tissue; and
a plurality of tethers each comprising a first portion configured to extend into the opening in the target tissue and a second portion configured to extend externally from the opening in the target tissue,
while the plurality of inflatable members is in an initial inflation state, the second portions of each tether being configured to extend through an opening of a corresponding inflatable member and move freely within the opening of the inflatable member, and
while the plurality of inflatable members is in a first inflation state more pressurized than the initial inflation state, the second portions of each tether being configured to extend through an opening of a corresponding inflatable member and at least a portion of the central portion of the corresponding inflatable member being configured to collapse around a corresponding portion of the tether.

Example 13: The system of any of the examples described herein, in particular example 12, wherein the opening of each of the plurality of inflatable members comprises a circular shape while the inflatable members are in the first inflation state.

Example 14: The system of any of the examples described herein, in particular example 12, wherein the opening of each of the plurality of inflatable members comprises an elongate shape while the inflatable members are in the first inflation state.

Example 15: The system of any of the examples described herein, in particular examples 12 to 14, wherein an externally oriented surface of the central portion of each of the plurality of inflatable members comprises a texture.

Example 16: The system of any of the examples described herein, in particular examples 12 to 15, wherein the central portion of each of the plurality of inflatable members defines a tubular lumen forming a cylindrical shape, the tubular lumen being configured to be perpendicularly oriented relative to the target tissue while the inflatable members are positioned over the target tissue, the openings of the inflatable members being a cross-sectional portion of a respective tubular lumen.

Example 17: A method, comprising:
providing an inflatable member over an opening in a target tissue, the inflatable member comprising:
a central portion defining an opening extending through the inflatable member, the opening of the inflatable member being configured to have the same orientation as the opening in the target tissue while the inflatable member is positioned over the target tissue;
positioning a plurality of tethers extending from the opening in the target tissue through the opening of the inflatable member; and
collapsing at least a portion of the central portion around portions of the plurality of tethers disposed within the opening of the inflatable member.

Example 18: The method of any of the examples described herein, in particular example 17, further comprising adjusting the tension of the plurality of tethers while respective portions of the plurality of tethers is disposed within the opening of the inflatable member and the at least a portion of the central portion is collapsed around the respective portions of the plurality of tethers.

Example 19: The method of any of the examples described herein, in particular example 17 or 18, further comprising:
inflating the inflatable member to a second inflation state, the second inflation state being more pressurized than an inflation state configured to allow tensioning of the plurality of tethers; and
tying a knot using a pair of tether tails of the plurality of tethers while respective portions of the plurality of tethers are disposed within the opening of the inflatable member in the second inflation state.

Example 20: The method of any of the examples described herein, in particular examples 17 to 19, wherein collapsing the at least a portion of the central portion comprises inflating the inflatable member to a first inflation state.

Example 21: The method of any of the examples described herein, in particular examples 17 to 19, wherein providing the inflatable member comprises providing the inflatable member in the first inflation state and a ring positioned within the opening of the inflatable member, and wherein collapsing the at least the portion of the central portion comprises removing the ring from the opening to allow at the least the portion of the central portion to collapse around the plurality of tethers.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in anyway required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the disclosure and claims below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms maybe interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A tether protection device, comprising:
an inflatable member configured to be positioned over an opening in a target tissue, the inflatable member comprising a central portion defining an opening extending through the inflatable member, the opening of the inflatable member being configured to allow for a plurality of tethers extending from the opening in the target tissue to be disposed at least partially therein; and
a fluid port extending through a wall portion of the inflatable member, the inflatable member being configured to be inflated via the fluid port to:
an initial inflation state in which the plurality of tethers is allowed to move freely within the opening of the inflatable member, and
a first inflation state in which at least a portion of the central portion is collapsed around and in contact with the plurality of tethers.

2. The device of claim 1, wherein an externally oriented surface of the central portion comprises a texture.

3. The device of claim 2, wherein the texture comprises at least one of a plurality of bumps and a plurality of ridges.

4. The device of claim 1, wherein a wall thickness of the central portion is less than a wall thickness of a circumferential portion of the inflatable member.

5. The device of claim 1, wherein a circumferential portion of the inflatable member forms a circular shape.

6. The device of claim 1, wherein the central portion defines a tubular lumen configured to be perpendicularly oriented relative to the target tissue while the inflatable member is positioned over the target tissue, the opening of the inflatable member being a cross-sectional portion of the tubular lumen.

7. The device of claim 6, wherein the tubular lumen forms a cylindrical shape.

8. The device of claim 1, wherein the opening of the inflatable member comprises a circular shape while the inflatable member is in the first inflation state.

9. The device of claim 1, wherein the opening of the inflatable member comprises an elongate shape while the inflatable member is in the first inflation state.

10. The device of claim 1, wherein the inflatable member is configured to be maintained in a second inflation state while a pair of tether tails of the plurality of tethers is tied together, the second inflation state being more pressurized than the first inflation state.

11. The device of claim 1, wherein the inflatable member is configured to be in the first inflation state and wherein the device further comprises a ring configured to be positioned in the opening of the inflatable member to maintain the opening and allow advancement therethrough of the plurality of tethers.

12. A suture system, comprising:
a plurality of inflatable members configured to be positioned over an opening in a target tissue, each of the plurality of inflatable members comprising:
a central portion defining an opening extending through the inflatable member, the opening of the inflatable member being configured to have the same orientation as the opening in the target tissue while the inflatable member is positioned over the target tissue; and
a plurality of tethers each comprising a first portion configured to extend into the opening in the target tissue and a second portion configured to extend externally from the opening in the target tissue,
while the plurality of inflatable members is in an initial inflation state, the second portions of each tether being configured to extend through an opening of a corresponding inflatable member and move freely within the opening of the inflatable member, and
while the plurality of inflatable members is in a first inflation state more pressurized than the initial inflation state, the second portions of each tether being configured to extend through an opening of a corresponding inflatable member and at least a portion of the central portion of the corresponding inflatable member being configured to collapse around a corresponding portion of the tether.

13. The system of claim 12, wherein the opening of each of the plurality of inflatable members comprises a circular shape while the inflatable members are in the first inflation state.

14. The system of claim 12, wherein the opening of each of the plurality of inflatable members comprises an elongate shape while the inflatable members are in the first inflation state.

15. The system of claim 12, wherein an externally oriented surface of the central portion of each of the plurality of inflatable members comprises a texture.

16. The system of claim 12, wherein the central portion of each of the plurality of inflatable members defines a tubular lumen forming a cylindrical shape, the tubular lumen being configured to be perpendicularly oriented relative to the target tissue while the inflatable members are positioned over the target tissue, the openings of the inflatable members being a cross-sectional portion of a respective tubular lumen.

17. A method, comprising:
providing an inflatable member over an opening in a target tissue, the inflatable member comprising:
a central portion defining an opening extending through the inflatable member, the opening of the inflatable member being configured to have the same orientation as the opening in the target tissue while the inflatable member is positioned over the target tissue;
positioning a plurality of tethers extending from the opening in the target tissue through the opening of the inflatable member; and
collapsing at least a portion of the central portion around portions of the plurality of tethers disposed within the opening of the inflatable member.

18. The method of claim 17, further comprising:
inflating the inflatable member to a second inflation state, the second inflation state being more pressurized than an inflation state configured to allow tensioning of the plurality of tethers; and
tying a knot using a pair of tether tails of the plurality of tethers while respective portions of the plurality of tethers are disposed within the opening of the inflatable member in the second inflation state.

19. The method of claim 17, wherein collapsing the at least a portion of the central portion comprises inflating the inflatable member to a first inflation state.

20. The method of claim 17, wherein providing the inflatable member comprises providing the inflatable member in the first inflation state and a ring positioned within the opening of the inflatable member, and wherein collapsing the at least the portion of the central portion comprises removing the ring from the opening to allow at the least the portion of the central portion to collapse around the plurality of tethers.
